(19) United States
(12) Patent Application Publication
(10) Pub. No.: US 2006/0259103 A1
(43) Pub. Date: Nov. 16, 2006

(54) Tissue ablation using multi-point convergent RF beams

(75) Inventor: Eric B. Stenzel, Tuam, Co. Galway (IE)

Correspondence Address: Vista IP Law Group LLP, 2040 MAIN STREET, 9TH FLOOR, IRVINE, CA, 92614, US

(73) Assignee: Boston Scientific Scimed, Inc., Maple Grove, MN (US)

(21) Appl. No.: 11/118,877

(22) Filed: Apr. 28, 2005

(51) Int. Cl. A61F 2/00

(52) U.S. Cl. 607/101

(57) ABSTRACT

A method of treating tissue includes delivering a first beam of radiofrequency energy towards target tissue, and delivering a second beam of radiofrequency energy towards the target tissue, wherein the first beam and the second beam intersect. A system for treating tissue includes a first radiofrequency energy source configured to deliver a first beam of radiofrequency energy, and a second radiofrequency energy source configured to deliver a second beam of radiofrequency energy, wherein the first radiofrequency energy source is aimed towards a first direction, and the second radiofrequency energy source is aimed towards a second direction that is different from the first direction.

### FIELD OF THE INVENTION

The field of the invention pertains to medical devices, and in particular, to systems and methods for ablating tissues.

### BACKGROUND

Tissue may be destroyed, ablated, or otherwise treated using thermal energy during various therapeutic procedures. Many forms of thermal energy may be imparted to tissue, such as radio frequency electrical energy, microwave electromagnetic energy, laser energy, acoustic energy, or thermal conduction.

In particular, radio frequency ablation (RFA) may be used to treat patients with tissue anomalies, such as liver anomalies and many primary cancers, such as cancers of the stomach, bowel, pancreas, kidney and lung. RFA treatment involves destroying undesirable cells by generating heat through agitation caused by the application of alternating electrical current (radio frequency energy) through the tissue.

Various RF ablation devices have been suggested for this purpose. For example, U.S. Pat. No. 5,855,576 describes an ablation apparatus that includes a plurality of wire electrodes deployable from a cannula or catheter. Each of the wires includes a proximal end that is coupled to a generator, and a distal end that may project from a distal end of the cannula. The wires are arranged in an array with the distal ends located generally radially and uniformly spaced apart from the catheter distal end. The wires may be energized in a monopolar or bipolar configuration to heat and necrose tissue within a precisely defined volumetric region of target tissue. The current may flow between closely spaced wire electrodes (bipolar mode) or between one or more wire electrodes and a larger, common electrode (monopolar mode) located remotely from the tissue to be heated. To assure that the target tissue is adequately treated and/or to limit damaging adjacent healthy tissues, the array of wires may be arranged uniformly, e.g., substantially evenly and symmetrically spaced-apart so that heat is generated uniformly within the desired target tissue volume.

When using the above described devices in percutaneous interventions, the cannula is generally inserted through a patient's skin, and the wires are deployed out of the distal end of the cannula to penetrate target tissue. The wires are then energized to ablate the target tissue. However, in some cases, it may not be possible to use such devices to treat certain tissues. For example, in the case of a brain tumor that is located deep within a brain, inserting the ablating wires into the brain may injure healthy brain tissues, thereby causing irreversible brain damage to the patient.

### SUMMARY

In accordance with some embodiments, a method of treating tissue includes delivering a first beam of radiofrequency energy towards target tissue, and delivering a second beam of radiofrequency energy towards the target tissue, wherein the first beam and the second beam intersect.

In accordance with other embodiments, a system for treating tissue includes a first radiofrequency energy source configured to deliver a first beam of radiofrequency energy, and a second radiofrequency energy source configured to deliver a second beam of radiofrequency energy, wherein the first radiofrequency energy source is aimed towards a first direction, and the second radiofrequency energy source is aimed towards a second direction that is different from the first direction.

In accordance with other embodiments, a method of providing radiofrequency energy includes providing a radiofrequency energy beam, expanding the radiofrequency energy beam, and focusing the expanded radiofrequency energy beam.

In accordance with other embodiments, a device for delivering energy towards tissue includes a microwave generator for generating a radiofrequency signal, a traveling wave tube for amplifying the radiofrequency signal to form a radiofrequency energy beam, a beam expander optic for expanding the radiofrequency energy beam, a collimating optic for collimating the expanded radiofrequency energy beam, and focusing optic for focusing the expanded radiofrequency energy beam.

Other and further aspects and features of the embodiments will be evident from reading the following detailed description of the preferred embodiments, which are intended to illustrate, not limit, the invention.

### BRIEF DESCRIPTION OF THE DRAWINGS

The drawings illustrate the design and utility of preferred embodiments. It should be noted that the figures are not drawn to scale and that elements of similar structures or functions are represented by like reference numerals throughout the figures. In order to better appreciate how the above-recited and other advantages and objects of the embodiments are obtained, a more particular description of the embodiments will be rendered, which are illustrated in the accompanying drawings.

FIG. 1 illustrates a block diagram of a tissue ablation system in accordance with some embodiments;

FIG. 2 illustrates a radiofrequency source in accordance with some embodiments;

FIG. 3 illustrates a method of treating tissue using the tissue ablation system of FIG. 1 in accordance with some embodiments; and

FIGS. 4A and 4B illustrate a block diagram of a tissue ablation system having six RF sources in accordance with some embodiments.

### DETAILED DESCRIPTION OF THE EMBODIMENTS

Various embodiments of the present invention are described hereinafter with reference to the figures. It should be noted that the figures are not drawn to scale and elements of similar structures or functions are represented by like reference numerals throughout the figures. It should also be noted that the figures are only intended to facilitate the description of specific embodiments. They are not intended as an exhaustive description of the invention or as a limitation on the scope of the invention. In addition, an aspect described in conjunction with a particular embodiment is not necessarily limited to that embodiment and can be practiced in any other embodiments.

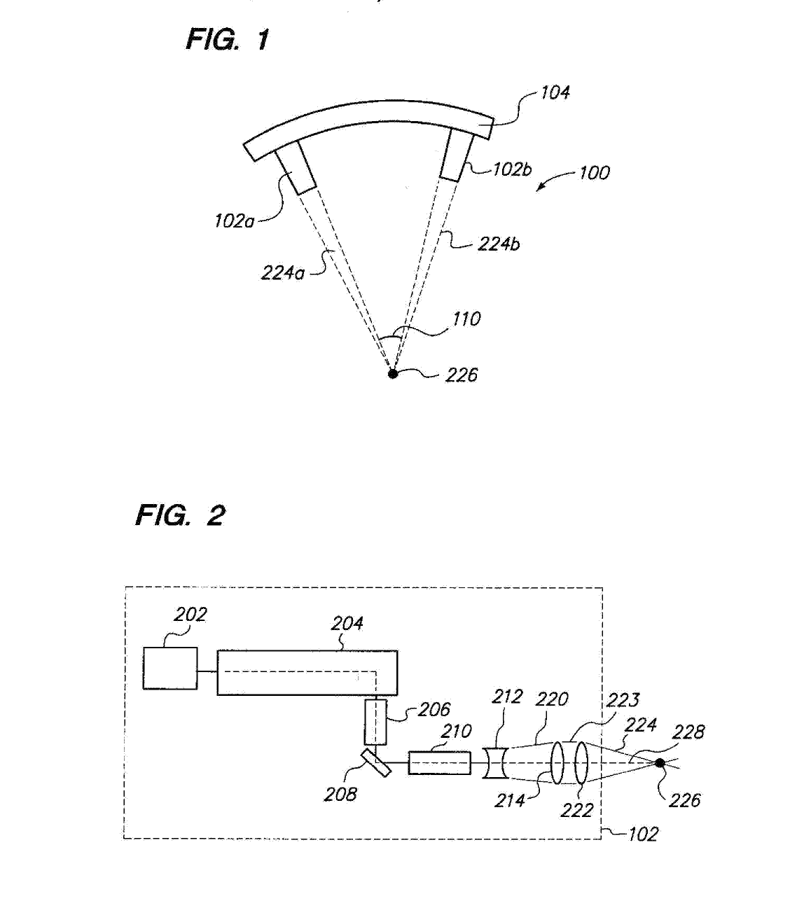

FIG. 1 illustrates a tissue ablation system 100 in accordance with some embodiments. The tissue ablation system 100 includes a first radiofrequency (RF) source 102a, a second RF source 102b, and a mounting structure 104 for securing the first and the second RF sources 102a, 102b relative to each other.

The first RF source 102a is configured to deliver a first beam 224a of RF energy, and the second RF source 102b is configured to deliver a second beam 224b of RF energy. The first and the second RF sources 102a, 102b are oriented at an angle relative to each other such that their respective beams 224a, 224b form an angle 110 and converge at a focal point 226. Although only two RF sources 102a, 102b are shown, in other embodiments, the tissue ablation system 100 can have more than two RF sources 102. For example, in other embodiments, the tissue ablation system 100 can further include a third RF source (not shown) configured to deliver a third beam of RF energy. In such cases, the third RF source is oriented at an angle relative to each of the first and the second RF sources 102a, 102b, such that the third beam of RF energy converge at the focal point 226. In other embodiments, the tissue ablation system 100 can include other numbers (e.g., 4 to 1000, or more) of RF sources 102.

The mounting structure 104 is not limited to the arch shape shown in the figure, and can have different shapes and configurations in different embodiments, as long as it provides a support to which the RF sources 102a, 102b can be secured. For example, in some embodiments, the mounting structure 104 can be a helmet or a headset configured to be placed on a patient's head. Alternatively, the mounting structure 104 can be a harness or a body-frame configured to be placed or worn by a patient. In the illustrated embodiments, the RF sources 102a, 102b are detachably secured to the mounting structure 104. For example, the mounting structure 104 can include a plurality of openings, and each of the RF sources 102a, 102b can include a screw for mating with one of the plurality of openings, thereby allowing each of the RF sources 102a, 102b to be selectively secured to the mounting structure 104 at different positions. Such configuration allows the position and/or orientation of the RF sources 102a, 102b to be adjusted. In other embodiments, the RF sources 102a, 102b can be slidably secured to the mounting structure 104 (e.g., using a guardrail or a tongue-and-groove connection, etc.), and/or rotatably secured to the mounting structure 104 (e.g., using a ball-joint connection, a shaft connection, etc.). In other embodiments, the system 100 can further include one or more positioners for dynamically adjusting the position of the RF sources 102 during a procedure. In further embodiments, the RF sources 102a, 102b can be fixedly secured to the structure 104 (e.g., using a weld connection, etc.). In other embodiments, the tissue ablation system 100 does not include the mounting structure 104. In such cases, one or both of the RF sources 102a, 102b can be held by a physician during use.

FIG. 2 illustrates one of the RF sources 102 in accordance with some embodiments. The RF source 102 includes a microwave generator 202, a traveling wave tube 204, a first waveguide 206, a RF reflector 208, a second waveguide 210, a beam expander optic 212, a collimating optic 214, and a focusing optic 222.

The microwave generator 202 is configured to supply a base RF signal. In the illustrated embodiments, the microwave generator 202 is a conventional RF power supply that operates at a frequency in the range from 100 MHz to 100 GHz, with a conventional sinusoidal or non-sinusoidal wave form. Such power supplies are available from many commercial suppliers, such as Valleylab, Aspen, Bovie, Richardson Electronics, and Agilent Technologies. In other embodiments, the microwave generator 202 can be configured to operate at different frequency ranges, and/or with different types of wave forms. Also, power supplies, such as GENLRS0.3 available from Cobermuegge of Norwalk, Conn., can also be used as the microwave generator 202.

The base RF signal supplied by the microwave generator 202 is fed through the traveling wave tube 204 to amplify the signal, thereby increase the energy of the signal. The amplified RF signal is directed through the first waveguide 206, which provides a means for the RF signal to travel without substantially diverging the signal. The RF signal travels through the first waveguide 206 to the RF reflector 208, which reflects the RF signal into the second waveguide 210. The second waveguide 210, like the first waveguide 206, also provides a means for the RF signal to travel without substantially diverging the signal. The RF reflector 208 can be, for example, a grid with line spacing in the order of the wavelength of the RF signal.

The reflected RF signal travels through the second waveguide 210 in a form of a RF beam, and into the beam expander optic 212. The beam expander optic 212, which may be, for example, a lens, is configured to expand or diverge the RF beam, such that the RF beam will have a desired characteristic (e.g., diameter) when striking the collimating optic 214. The beam expander optic 212 can be implemented using known optic devices or techniques.

The diverging RF beam 220 is then passed through the collimating optic 214, which collimates the RF beam 220 (e.g., prevents the RF beam from further diverging). The collimated RF beam 223 is then passed through the focusing optic 222, which focus the collimated beam 223 into the focal point 226. The collimating optic 214 and the focusing optic 222 can be implemented using lenses or any of the known optical devices. U.S. Pat. Nos. 4,337,759 and 5,577,492 disclose optical devices that can be used to implement the collimating optic 214 and the focusing optic 222. In other embodiments, the collimating optic 214 and/or the focusing optic 222 can be implemented using microwave optic technology, which allows a bending of a beam traveling therethrough to be controlled. This has the advantage of changing a shape of the beam such that the generated beam conforms to a shape of a target tissue.

In the illustrated embodiments, the focal point of each of the RF sources 102 can be anywhere between 1 to 30 inches from the collimating optic 214. Such feature is suitable for treating a variety of tissue at different bodily location, such as brain tumors or cancers. In other embodiments, the focal zone of each of the RF sources 102 can be at other distances (e.g., at infinity) from the collimating optic 214. Also, in further embodiments, the range of distances between one RF source (e.g., RF source 102a) can be different from the range of distances between another RF source (e.g., RF source 102b). In some embodiments, the focusing optic 222, and/or the collimating optic 214 can be positioned along an axis 228, thereby allowing a distance between the optics 214, 222, and a position of the focal point 226, to be adjusted. For example, the RF source 102 can further include a positioner secured to the focusing optic 222 for moving the focusing optic 222, and/or a positioner secured to the collimating optic 214 for moving the collimating optic 214. In further embodiments, the RF source itself can be positioned (e.g., by a positioner) to thereby adjust a position of the focal point 226.

In other embodiments, instead of each RF source 102 having its own microwave generator 202 and traveling wave tube 204, two or more RF sources 102 can share a common microwave generator 202 and/or traveling wave tube 204. For example, in other embodiments, the traveling wave tube 204 can include a beam splitter, which divides the base RF signal supplied by the microwave generator 202 into a plurality of RF beams. The beam splitter can be implemented using a RF reflector block or any of the known optical devices. As used in this specification, the term “plurality” refers to a number that is more than one, such as two or more (e.g., 1000). Also, instead of having two waveguides 206, 210, in other embodiments, the RF source 102 can include only one waveguide, or more than two waveguides.

It should be noted that the RF source 102 is not limited to the example described, and that in other embodiments, the tissue ablation system 100 can use other RF sources having different configurations.

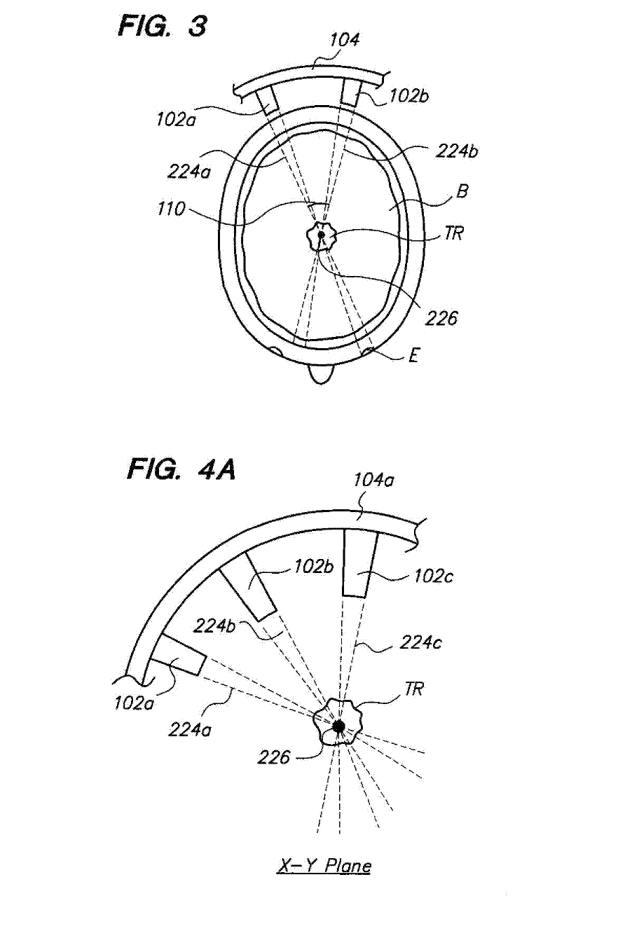

Referring now to FIG. 3, the operation of the tissue ablation system 100 will now be described. First, a target tissue region is determined. This can be accomplished using any of the known conventional techniques. For example, a MRI or CT scan can be performed on a patient. The result of the scan is then used to identify a target tissue region TR. The target tissue region TR can be, for example, a tumor or a cancer, which is desired to be ablated. As shown in the figure, the target tissue region TR is a tumor within a brain B. However, it should be understood by those skilled in the art that the system 100 can be used to treat tissue at other locations within a patient's body.

Next, a treatment plan is determined. In the illustrated embodiments, this involves determining an orientation and a position for each of the RF sources 102, such that the RF sources 102 can deliver RF beams that are aimed towards the target tissue region TR. In addition, an energy level of each of the converging RF beams 224 is also determined to ensure that the combined energy level at the target tissue region TR where the RF beams 224 intersect is at or above a prescribed threshold. For example, the prescribed threshold can be selected to ensure that the combined energy level is sufficient for ablating the target tissue region TR.

In some embodiments, in addition to considering targeted tissue region TR, non-targeted tissue upstream from the target tissue region TR (tissue between each of the RF sources 102 and the target tissue region TR) are also considered when determining the treatment plan. In particular, the position, orientation, and/or an energy dose to be delivered by each of the RF sources 102 is determined such that the RF sources 102 can deliver RF beams that are aimed towards the target tissue region TR, while protecting non-targeted tissue that are upstream from the target tissue region TR. Such can be accomplished, for example, by ensuring that each of the RF beam traversing non-targeted tissue that is upstream to the target tissue region TR has an energy that is below a prescribed threshold for protecting the non-targeted tissue.

In other embodiments, instead of, or in addition to, considering the non-targeted tissue that are upstream from the target tissue region TR, non-targeted tissue downstream from the target tissue region TR (tissue through which RF beam exiting the target tissue region TR travels) are also considered when determining the treatment plan. In particular, the position, orientation, and/or an energy dose to be delivered by each of the RF sources 102 is determined such that the RF sources 102 can deliver RF beams that are aimed towards the target tissue region TR, while protecting non-targeted tissue that are downstream from the target tissue region TR. Such can be accomplished, for example, by ensuring that each of the RF beam traversing non-targeted tissue that is downstream to the target tissue region TR has an energy that is below a prescribed threshold for protecting the non-targeted tissue. For example, as shown in FIG. 3, the RF source 102a is positioned and oriented such that RF beam 224a exiting the target tissue region TR is directed towards the patient's eye E. In such cases, if desired, the RF source 102a may be repositioned, and/or the energy level of the RF beam 224a may be adjusted, to protect the patient's eye E from being injured by RF energy (e.g., to prevent, or at least reduce, the possibility of cataract formation).

In some embodiments, determining the treatment plan also includes determining RF energy wavelength and phase for each of the RF sources 102 to ensure that each RF energy beam is in phase with other RF beam(s) at the target tissue region, thereby maximizing energy to be delivered to the target tissue region while minimizing energy at non-target tissue region surrounding the target tissue region. Each RF energy beam can be frequency modulated to provide general phase alignment. By way of non-limiting example, each RF energy beam could be 2.45 GHz and oscillate by at least ±50 MHz where the oscillation cycle could be every 10 seconds.

In the above embodiments, the RF sources 102 are activated simultaneously. Alternatively, the RF sources 102 can be activated in sequence. For example, in some embodiments, the RF source 102a is activated for a prescribed duration (e.g., 1 microsecond) to emit RF energy while the RF source 102b is not activated. Afterwards, the RF source 102b is then activated for a prescribed duration while the RF source 102a is not activated. In other embodiments, the RF source 102b can be activated to deliver a sequence of pulses of RF energy while the RF source 102b is not activated. Afterwards, the RF source 102b is then activated to deliver a sequence of pulses of RF energy while the RF source 102a is not activated.

In the illustrated embodiments, before the RF sources 102 deliver RF beams 224, the RF sources 102 are configured to ensure that each of the RF sources 102 will provide a RF beam having a desired characteristic. For example, the beam expander optic 212, the collimating optic 214, and/or the focusing optic 222 of each of the RF sources 102 is configured to ensure that it will provide a RF beam at a desired energy level as specified by the treatment plan. For example, lens having certain densities, surface profiles, and/or other diffraction properties, can be selected to be used in the RF sources 102. In some embodiments, the beam expander optic 212, the collimating optic 214, and/or the focusing optic 222 of each of the RF sources 102 are positionable along the axis 228 of the RF source 102, thereby allowing each of the RF sources 102 be configured to provide a desired RF beam 224 (e.g., a RF beam having an energy level that is within a prescribed range).

Next, the RF sources 102 are positioned relative to the target tissue region TR in accordance with that prescribed by the treatment plan, and the RF sources 102 are activated to deliver a plurality of RF beams 224 towards the target tissue region TR. In the illustrated embodiments, the RF beams 224 are continuous beams. Using continuous beams allow more energy to be delivered to the target tissue region, thereby allowing completion of a procedure in a shorter period. Alternatively, the RF beams 224 can be pulsed. Using pulsed RF beams allows energy absorbed in the non-targeted tissue to be dissipated in between pulses (where blood or other fluid flow provides a cooling effect), thereby preventing generation of excessive heat in non-targeted tissue that are adjacent to the target tissue. If the beam expander optic 212 and the focusing lens system 214 are positionable, one or both of them can be positioned during a treatment to modulate the RF beams 224, e.g., to provide RF beams 224 having certain shapes, focal distances, and/or energy densities. As a result of delivering the plurality of RF beams 224 towards the target tissue region TR, the region TR is necrosed, thereby creating a lesion on the treatment region TR.

As can be appreciated by those skilled in the art, delivering RF energy from different positions around the target region TR increases the surface area of the patient's skin through which RF beam energy from the RF sources 102 is passing. This, in turn, prevents, or at least reduces the risk of, excessive energy density at a patient's skin and at non-targeted tissue, thereby preventing injury to the patient's skin and the non-targeted tissue (at upstream and/or downstream from the target tissue region TR).

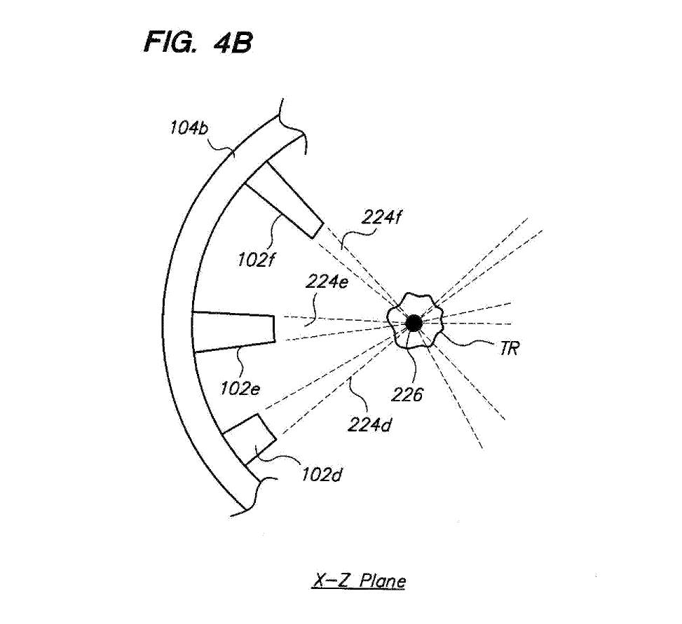

In the above embodiments, the RF sources 102a, 102b are oriented such that their respective RF beams 224a, 224b form a first plane. However, in other embodiments, one or more additional RF source (e.g., 102c, etc.) can be provided such that its RF beam lies approximately within the first plane. Also, in other embodiments, one or more additional RF source can be provided and be oriented such that its RF beam forms a second plane with another RF beam, wherein the second plane forms an angle with the first plane. FIGS. 4A and 4B shows a tissue ablation system having six RF sources 102a-102f. The RF sources 102a-102c are positioned and oriented such that their RF beams 224a-224c approximately lie within a first plane (e.g., the X-Y plane), and the RF sources 102d-102f are positioned and oriented such that their RF beams 224d-224f approximately lie within a second plane (e.g., the X-Z plane). The RF beams 224a-224f intersect each other at the target tissue region TR to thereby create an ablation zone at the target tissue region TR. In the illustrated embodiments, the first plane is 90° from the second plane. However, in other embodiments, the first plane can be at other angles relative to the second plane.

Although the above method has been described with reference to placing the RF sources 102a, 102b outside a patient, the scope of the invention should not be so limited. In other embodiments, one or more RF sources 102 can be placed at least partially within a patient. For example, in some embodiments, the RF source 102a is positioned external to a patient, and the RF source 102b is placed internal to the patient. In such cases, the RF source 102a delivers a first RF beam from outside the patient to a target region within the patient, while the RF source 102b delivers a second RF beam from within the patient to the target region. In other embodiments, both RF sources 102a, 102b are placed at least partially within a patient, such that RF beams are delivered from the RF sources 102a, 102b from within the patient towards a target region.

Although the tissue ablation system 100 has been described with reference to tissue ablation, in other embodiments, the system 100 can be used to perform other medical procedures. For example, in other embodiments, the system 100 can be used as a RF energy scalpel or cutting tool to cut tissue within a body. Such may help relieve internal fluid pressure that has been built-up due to a contusion to the head of a patient, or due to a rupture of an aneurysm. For example, the system 100 can be used to create a path for allowing fluid to drain from one location to another location within a patient. In some cases, the system 100 can also be used to open up a vein that is close to an aneurysm, thereby allowing accumulated fluid to drain through the venous system. In other embodiments, the system 100 can also be used to break apart other substance within a patient's body. For example, in some cases, the system 100 can be used to break apart an emboli or a deposit. In such cases, an embolic protection device may be placed downstream from the lesion for catching the emboli or deposit after it has been broken apart. The system 100 can also be used with other technologies in other embodiments.

Although particular embodiments have been shown and described, it should be understood that the above discussion is not intended to limit the present invention to these embodiments. It will be obvious to those skilled in the art that various changes and modifications may be made without departing from the spirit and scope of the present invention. The present invention is intended to cover alternatives, modifications, and equivalents that may fall within the spirit and scope of the present invention as defined by the claims.

What is claimed:

1. A method of treating tissue, comprising:
delivering a first beam of radiofrequency energy towards target tissue; and
delivering a second beam of radiofrequency energy towards the target tissue;
wherein the first beam and the second beam intersect.

2. The method of claim 1, wherein the first beam and the second beam intersect at the target tissue.

3. The method of claim 1, wherein the steps of delivering are performed simultaneously.

4. The method of claim 1, wherein the delivering the first beam comprises emitting radiofrequency energy in pulses.

5. The method of claim 1, wherein the intersected beam has an energy level sufficient for ablating the target tissue.

6. The method of claim 1, further comprising delivering a third beam of radiofrequency energy towards the target tissue, such that the first, second, and third beams intersect.

7. The method of claim 6, wherein the intersected beam has an energy level sufficient for ablating the target tissue.

8. The method of claim 1, further comprising:
delivering a third beam of radiofrequency energy towards the target tissue; and
delivering a fourth beam of radiofrequency energy towards the target tissue;
wherein the first and the second beam form a first plane, the third and the fourth beam form a second plane, and the first plane forms an angle with the second plane.

9. A system for treating tissue, comprising:
a first radiofrequency energy source configured to deliver a first beam of radiofrequency energy; and
a second radiofrequency energy source configured to deliver a second beam of radiofrequency energy;
wherein the first radiofrequency energy source is aimed towards a first direction, and the second radiofrequency energy source is aimed towards a second direction that is different from the first direction.

10. The system of claim 9, wherein the first and the second directions are selected such that the first and the second beams intersect each other.

11. The system of claim 9, further comprising a third radiofrequency energy source configured to deliver a third beam of radiofrequency energy, wherein the third radiofrequency energy is aimed towards a third direction that is different from the first and the second directions.

12. The system of claim 11, wherein the first, second, and third beams intersect each other.

13. The system of claim 11, further comprising a fourth radiofrequency energy source configured to deliver a fourth beam of radiofrequency energy, wherein the fourth radiofrequency energy is aimed towards a fourth direction, the first and the second beams form a first plane, the third and the fourth beams form a second plane, and the first plane forms an angle with the second plane.

14. The system of claim 9, wherein the first radiofrequency energy source comprises:
a microwave generator for generating a radiofrequency signal;
a traveling wave tube for amplifying the radiofrequency signal to form a radiofrequency energy beam;
a beam expander optic for expanding the radiofrequency energy beam;
a collimating optic for collimating the expanded radiofrequency energy beam; and
a focusing optic for focusing the expanded radiofrequency energy beam.

15. The system of claim 14, wherein a distance between the collimating optic and the focusing optic is adjustable.

16. The system of claim 9, wherein the first radiofrequency energy source and the second radiofrequency energy source are structurally connected to each other.

17. The system of claim 9, wherein the first radiofrequency energy source and the second radiofrequency energy source are moveable relative to each other.

18. The system of claim 17, further comprising a positioner for moving one or both of the first and the second radiofrequency energy source.

19. A method of providing radiofrequency energy, comprising:
providing a radiofrequency energy beam;
expanding the radiofrequency energy beam; and
focusing the expanded radiofrequency energy beam.

20. The method of claim 19, wherein the providing comprises:
generating a radiofrequency signal;
amplifying the radiofrequency signal; and
directing the amplified radiofrequency signal to a reflective surface.

21. The method of claim 20, wherein the reflective surface comprises a grid with line spacing approximately equals to a wavelength of the amplified radiofrequency signal.

22. A device for delivering energy towards tissue, comprises:
a microwave generator for generating a radiofrequency signal;
a traveling wave tube for amplifying the radiofrequency signal to form a radiofrequency energy beam;
a beam expander optic for expanding the radiofrequency energy beam;
a collimating optic for collimating the expanded radiofrequency energy beam; and
a focusing optic for focusing the expanded radiofrequency energy beam.

23. The device of claim 22, further comprising a reflective surface in operative association with the traveling wave tube and the beam collimator.

24. The device of claim 23, wherein the reflective surface comprises a grid with line spacing approximately equals to a wavelength of the amplified radiofrequency signal.

25. The device of claim 22, wherein a distance between the collimating optic and the focusing optic are adjustable.
